(19) United States
(12) Patent Application Publication
(10) Pub. No.: US 2022/0412946 A1
(43) Pub. Date: Dec. 29, 2022

(54) SYSTEM, METHOD, AND APPARATUS FOR PREDICTING PHYSICAL PROPERTIES BY INDIRECT MEASUREMENT

(71) Applicant: VALIDERE TECHNOLOGIES INC., Toronto (CA)

(72) Inventors: Ian Bruce BURGESS, Toronto (CA); John NGUYEN, Etobicoke (CA); Nicholas Ainsley DUCILLE, Ajax (CA); Tingkai ZHANG, Toronto (CA); Patrick James ROBINSON, Toronto (CA)

(73) Assignee: VALIDERE TECHNOLOGIES INC., Toronto, ON (CA)

(21) Appl. No.: 17/784,962

(22) PCT Filed: Jul. 2, 2020
(86) PCT No.: PCT/CA2020/050920

§ 371 Date: Jun. 13, 2022

Related U.S. Application Data
(60) Provisional application No. 62/947,793, filed on Dec. 13, 2019.

(51) Int. Cl. G01N 33/28

(52) U.S. Cl. CPC G01N 33/28

(57) ABSTRACT

According to at least one exemplary embodiment, a system, method, and apparatus for predicting physical properties by indirect measurement is disclosed. The system, method, and apparatus provide for measurement of physical properties of petroleum products by calculating predictions of the properties based on diverse inputs. These predictions may be updated in real time and may be used to modify various processes in midstream and downstream petroleum operations. Various use cases for the system, method, and apparatus for predicting physical properties by indirect measurement are disclosed.

### CROSS-REFERENCE TO RELATED APPLICATION

This application is the national phase filing of PCT/CA2020/050920, which claims priority to U.S. Provisional Patent Application Ser. No. 62/947,793, filed Dec. 13, 2019, the contents of which are hereby incorporated by reference in their entirety.

### BACKGROUND

Midstream and downstream petroleum operations necessitate measurement of physical properties of various petroleum products along multiple points. Measurements are performed, for example, to ensure desired proportions of various petroleum products in the blends thereof, to determine the concentrations of undesired constituents in the petroleum products, to comply with regulations as to various properties of the petroleum products, and so forth. Typically, the measurements are performed by physical analyzing equipment (a “physical analyzer”) that is situated in-line at necessary locations, as well as by manually retrieving samples at the locations and transferring them to an on-site or off-site laboratory for analysis. However, in many situations, providing physical analyzers or performing laboratory analysis is impractical or cost-prohibitive. An alternate solution to obtain desired measurements of physical properties of petroleum products is therefore desired.

### SUMMARY

According to at least one exemplary embodiment, a system, method, and apparatus for predicting physical properties by indirect measurement is disclosed. The embodiments disclosed herein allow for measurement of physical properties of petroleum products by calculating predictions of the properties based on diverse inputs. These predictions may be updated in real time and may be used to modify various processes in midstream and downstream operations.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary embodiment of a system, method, and apparatus for predicting physical properties by indirect measurement.

FIG. 2 shows an exemplary use case for a system, method, and apparatus for predicting physical properties by indirect measurement.

FIG. 3 shows another exemplary use case for a system, method, and apparatus for predicting physical properties by indirect measurement.

FIG. 4 shows yet another exemplary use case for a system, method, and apparatus for predicting physical properties by indirect measurement.

FIG. 5 shows yet another exemplary use case for a system, method, and apparatus for predicting physical properties by indirect measurement.

FIG. 6 shows yet another exemplary use case for a system, method, and apparatus for predicting physical properties by indirect measurement.

FIG. 7 shows yet another exemplary use case for a system, method, and apparatus for predicting physical properties by indirect measurement.

FIG. 8 shows yet another exemplary use case for a system, method, and apparatus for predicting physical properties by indirect measurement.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

According to at least one exemplary embodiment, a method, system, and apparatus for predicting physical properties by indirect measurement is disclosed, and referred to herein as a “virtual analyzer” for brevity purposes.

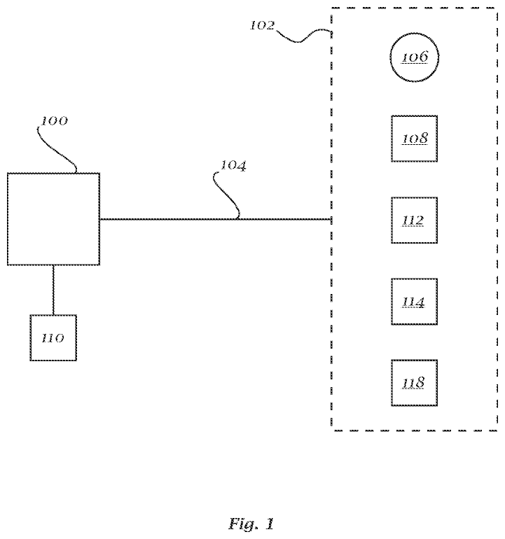

As shown in FIG. 1, the virtual analyzer 100 is configured to perform a prediction of one or more physical properties of a petroleum product without the requirement that the physical property be measured directly in a particular location. Generally, the virtual analyzer 100 can perform these predictions by receiving various measurements from a plurality of inputs 102, and utilizing those measurements to calculate a quality parameter of the substance as if it were physically measured at the particular location.

Furthermore, the prediction calculated by the virtual analyzer may be a “live”, or real-time prediction, as discussed further below. The real-time prediction can be derived from a model that can take into account multiple sources of data. For example, the sources of data may be one or more sources of real-time data 104 being received from one or more in-line meters 106 disposed throughout a location, such as a processing or transport facility. This real-time data may include flow, density, temperature, pressure, and so forth. This real-time data may further include data from in-line physical analyzers for various properties such as vapor pressure, sulfur content, and so forth. The sources of data may also be one or more lab samples 108, which may include both samples taken from composite or proportional samplers, or spot samples that are analyzed in off-site labs by third parties, or in on-site labs by field personnel.

The virtual analyzer can collect and aggregate the data received from the multiple sources of data. To this end, the virtual analyzer can include: a database 110 where received measurement and analysis data is stored; a plurality of field devices 112, such as portable computing or communications devices, which include software for recording the time and location of substance samples and sample laboratory analyses; Laboratory Information Management Systems 114 (“LIMS”) that can retrieve field laboratory data from instruments; internet-enabled devices that are connected to portable physical analyzers 118 and that are configured to communicate analysis results to the database of the virtual analyzer; and so forth.

The predictions calculated by the virtual analyzers are “live” or real-time predictions in the sense that such predictions are made at a minimum frequency. Generally, the minimum frequency of predictions can be configured to be such that the predictions calculated by the virtual analyzer are calculated sufficiently frequently so as to allow the predictions to be used to make alterations to the parameters of a particular process during the execution of the process. In certain embodiments, the process is a blending process, where two or more crude and/or refined petroleum liquids (e.g. crude oils, natural gas liquids, distillates, refinery intermediate products, refined petroleum that meet sales specifications for gasoline, diesel, jet fuel, fuel oil, chemical products, etc.) are combined to form a mixture that meets the specifications for a specific crude oil grade (e.g. WTI, DSW, WTL, Brent, WCS, MSW, CRW, MSB, Midale, PCH, Mars, etc.). In certain embodiments, the process is blending of two or more crude and/or refined petroleum products to meet the specification for a refined petroleum product (e.g. gasoline, diesel, jet fuel, fuel oil, chemical products, etc.). For many blending processes, the value of the final product relative to the blend feedstocks is maximized when one or more specific physical properties (e.g. density, vapour pressure, viscosity, flash point, pour point, distillation profile, compatibility, etc), or aspects of chemical composition (e.g. sulphur content, vanadium content, nickel content, asphaltene content, hydrogen sulphide content, heat of combustion, etc) are tuned as close as possible to specific optimal values. A blending system that uses feedback from virtual analyzer readings of these optimization parameters to adjust blend ratios in response to compositional changes in feedstocks can achieve properties of the overall blend that is closer to the optimal specifications. In certain embodiments, the process consists of a separation that adjusts the physical properties and/or chemical composition of the petroleum liquid by removing one or more components of the petroleum mixture. Examples of separation processes include free-water knockout, treatment processes to remove dissolved or emulsified water from petroleum liquids, heat treatment to remove light ends (e.g. methane, ethane, propane, butanes, etc) from crude oils, distillation processes that separate petroleum mixtures according to boiling point range. In certain embodiments, the process performs a chemical modification to one or more components of a petroleum liquid. Examples of chemical modifications include hydrogen sulphide scavenging, cracking, coking, desulphurization, aromatization, etc. As with blending processes, the value of the final product relative to the separation or chemical process feedstocks is maximized when one or more specific physical properties (e.g. density, vapour pressure, viscosity, flash point, pour point, distillation profile, compatibility, etc), or aspects of chemical composition (e.g. sulphur content, vanadium content, nickel content, asphaltene content, hydrogen sulphide content, heat of combustion, etc) are tuned as close as possible to specific optimal values. A system performing separation or chemical modification that uses feedback from virtual analyzer readings of these optimization parameters to adjust process parameters (e.g. process temperature, reagent concentrations, etc.) in response to compositional changes in feedstocks can achieve properties of the overall composition that is closer to the optimal specifications.

More specifically, in some embodiments, the minimum frequency of the predictions can be in the range between once every three hours to once every five minutes. More specifically, in some embodiments, the minimum frequency may be once every 5 minutes, once every 10 minutes, once every 20 minutes, once every 30 minutes, once every 60 minutes, once every 90 minutes, once every 120 minutes, once every 180 minutes, and so forth. In yet other embodiments, the minimum frequency may be adjusted as desired. In some blending applications, the minimum frequency of predictions is correlated with the timescale on which the composition of the feedstocks changes. For example, in embodiments where one or more feedstock enters the blending infrastructure from a truck offload, the minimum frequency of predictions must allow for at least one measurement per truck. In embodiments where one or more feedstock enters the blend apparatus from a tank, the minimum frequency of predictions must be shorter than the transit time of a typical molecule through the tank. The average transit time can be determined from the flow rates into and out of the tank and the tank levels. In some embodiments where the virtual analyzer is used to tune a separation or chemical modification process, the minimum frequency of predictions should match the minimum frequency that the process parameters can be modified to produce a useful effect. For example, in embodiments where hydrogen sulphide scavenger is added to crude oil before shipping to neutralize hydrogen sulphide contained in the oil, the minimum frequency of predictions must be at least once per batch. In some embodiments where shipments are delivered from a tank, the minimum frequency of predictions should match the transit time of a liquid molecule through the tank. In some embodiments where the hydrogen sulphide scavenger is added before the oil is loaded onto a truck, the minimum frequency of predictions should at least match the time taken to load one truck.

In some embodiments the calculations behind a virtual analyzer prediction consist of a combination of physical models that map out the process being performed (process models) and physical and statistical models that determine relevant physical and chemical properties of the inputs and output of the process (property models), with the process model incorporated into the calculation of the outputs. For example, in a blending process, the process model determines the exact ratios of components that were blended over time, while the property model predicts the properties of the input streams and the blended output stream. In separation or chemical modification processes, the process model may include calculations that rely on physical and chemical equations to determine the relationship between process parameters and output properties, given a known input composition. In some embodiments, the process model may also include statistical predictions of how the composition of the liquid changes in response to process parameters using historical data. In some embodiments, an additional feedback loop is used predict the optimal schedule to take laboratory tests to produce the most useful input data for the virtual analyzer model. In some embodiments, statistical analysis of the correlation between measured parameters is further used to determine when further laboratory testing should be done. In some embodiments, this information is fed directly to alert operators (e.g. on the control panel or via email, SMS, etc.) when to take samples to send to the laboratory. In some embodiments, these alerts go directly to laboratory personnel.

As an example, one exemplary embodiment of a virtual analyzer can include a virtual analyzer for calculating the vapor pressure of a blended stream of crude oil. As shown in FIG. 1, the virtual analyzer can receive inputs from data sources such as density analyzers and laboratory samples. The data received from the density analyzers can include, for example, the density and flow rate measured for each of two streams of crude oil. The data received from laboratory samples can include the distribution of vapor pressure and density of each of two streams of crude oil. The virtual analyzer can utilize this data to calculate the vapor pressure of the blended stream from the two streams of crude oil. In some embodiments, the calculation may include a statistical estimation of the vapour pressure of each incoming stream based on the live density readings from the density analyzer and historical records of the density and vapour pressure of these streams from a combination of laboratory samples and density analyzer readings. In some embodiments, the vapour pressure of the blended stream is calculated from the estimated stream values using a process model that incorporates mass balance, thermodynamic relationships (e.g. Raoult's law) and estimates of composition changes occurring during and/or after the blend (e.g. mixing, tank evaporation, sample stratification over time, etc.).

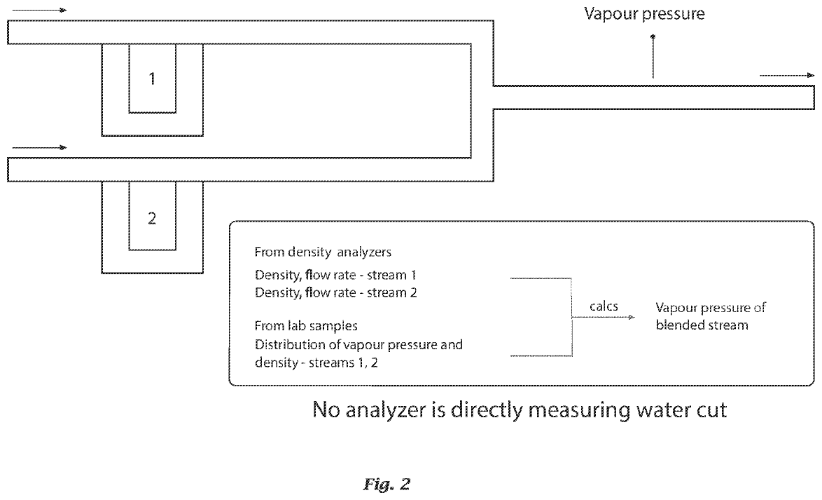

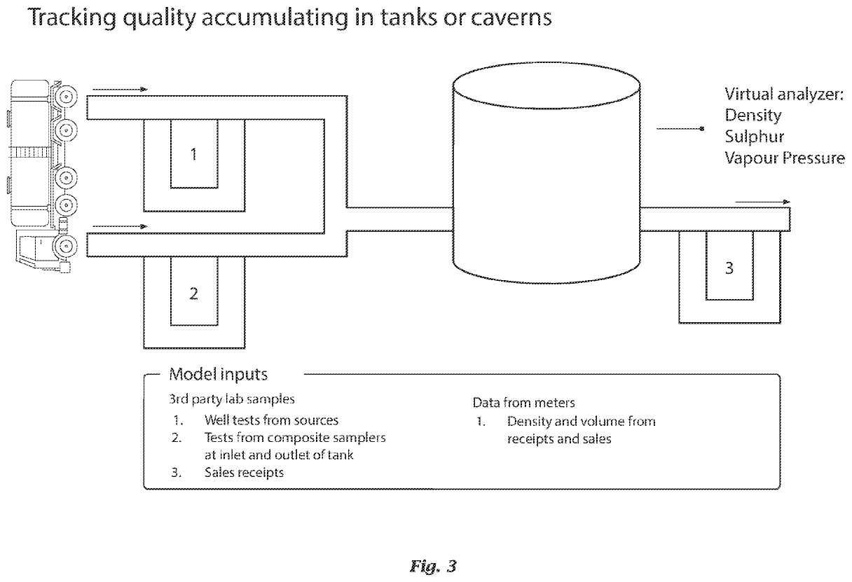

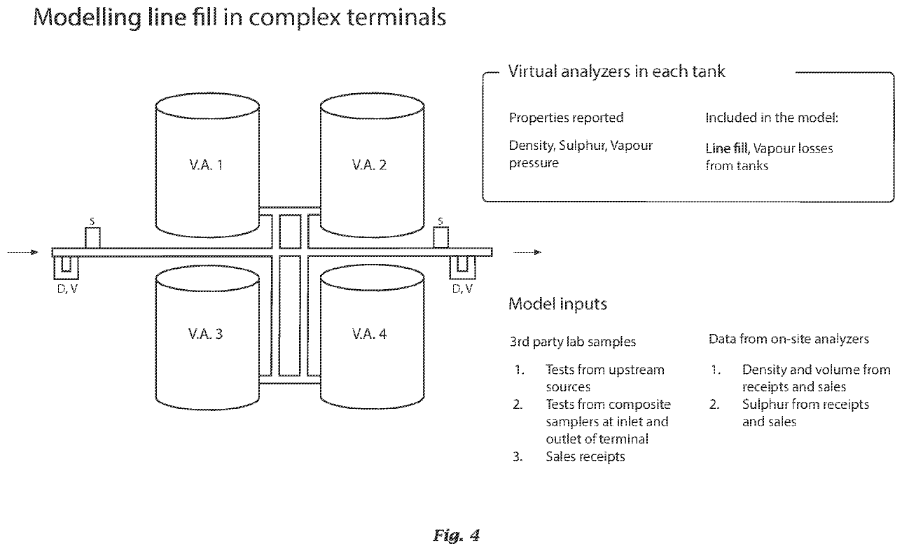

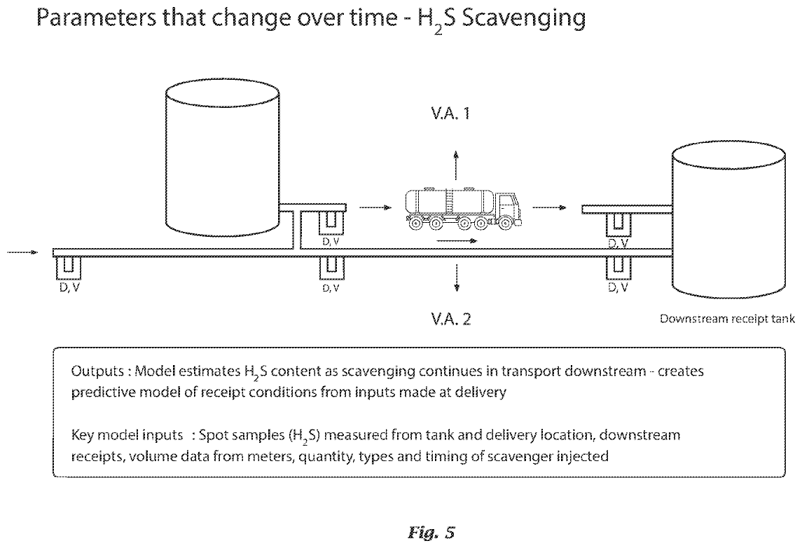

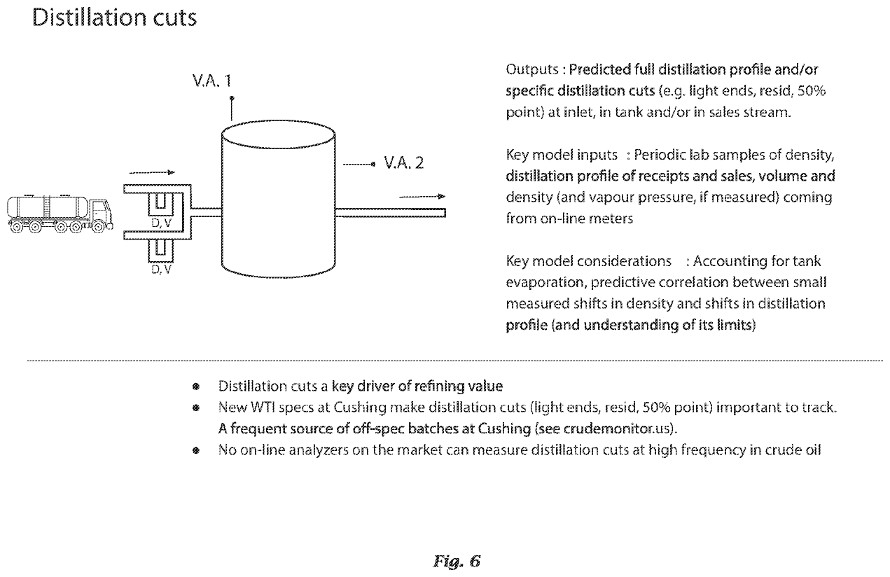

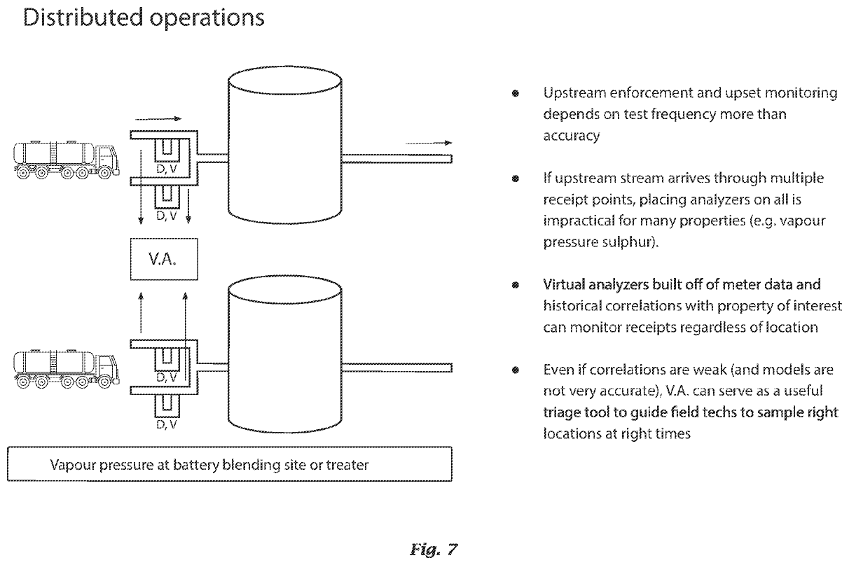

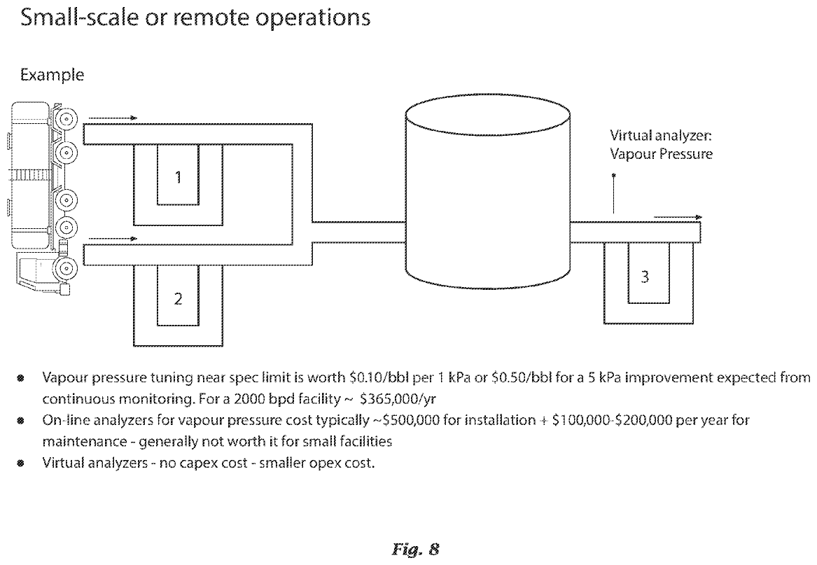

Exemplary advantageous use cases for virtual analyzers can include situations where quality tracking is needed in inconvenient locations, for example, inside caverns, storage tanks, and the like; measuring parameters that do not have practical physical analyzers, such as total acid number (TAN), distillation cuts, viscosity, asphaltene solubility and product compatibility, refined product specifications such as octane rating, etc.; and locations where physical analyzers are cost-prohibitive, for example, remote locations, small terminals, and the like. As further examples, several exemplary embodiments of use cases for virtual analyzers are shown in FIGS. 2-8.

In some exemplary embodiments, predictions of the virtual analyzer may be utilized to change the parameters of a process in real time. The process may be, for example, a blending process of various petroleum products. The petroleum products can include crude oil, NGLs, condensate and/or refined petroleum products. The parameters of the blend, for example the blend ratio, may be adjusted in real time based on live predictions from the virtual analyzer.

As an example, a blend ratio may be determined and adjusted based on several sources of data. Such data may be: the vapor pressure of a first petroleum product that is entering a blending unit; the vapor pressure of a second petroleum product that is entering a blending unit; and the desired vapor pressure of the resultant blend. Alternatively, such data may be: the vapor/liquid ratio of a first petroleum product that is entering a blending unit; the vapor/liquid ratio of a second petroleum product that is entering a blending unit; and the desired vapor/liquid ratio of the resultant blend. This data may be provided as a real time prediction by the virtual analyzer based on the various data inputs received by the virtual analyzer.

More specifically, in various exemplary embodiments, virtual analyzers may be used for at least the following use cases. In each use case, virtual analyzer calculations may rely on a combination of process models and property models of the input and output streams, as described above. Examples of input data most relevant to the predictions are listed next to each example.

In one exemplary embodiment of a virtual analyzer, vapor pressure may be predicted for the blending of butane into crude oil. In this embodiment, input data may include historical laboratory measurements of the density and vapour pressure of the crude oil stream, compositional analysis of the butane stream (isobutane:n-butane ratio, residual propane and ethane content), and flow rate and density readings from in-line analyzers measuring both streams. In some embodiments, statistical models are used to estimate the live vapor pressure of the crude and butane streams before the blend.

In another exemplary embodiment of a virtual analyzer, vapor pressure may be predicted for the blending of butane into condensate. In this embodiment, input data may include historical laboratory measurements of the density and vapour pressure and/or compositional analysis (concentration of methane, ethane, propane, butanes, pentanes, inert gases, average molecular weight) of the condensate stream, compositional analysis of the butane stream (isobutane:n-butane ratio, residual propane and ethane content), and flow rate and density readings from in-line analyzers measuring both streams. In some embodiments, statistical models are used to estimate the live vapor pressure of the condensate and butane streams before the blend.

In yet another exemplary embodiment of a virtual analyzer, vapor pressure may be predicted for the blending of butane into gasoline. In this embodiment, input data may include historical laboratory measurements of the density and vapour pressure and/or compositional analysis (concentration of methane, ethane, propane, butanes, pentanes, inert gases, average molecular weight) of the gasoline stream, compositional analysis of the butane stream (isobutane:n-butane ratio, residual propane and ethane content), and flow rate and density readings from in-line analyzers measuring both streams. In some embodiments, statistical models are used to estimate the live vapor pressure of the gasoline and butane streams before the blend.

In yet another exemplary embodiment of a virtual analyzer, flash point may be predicted for the blending of butane into gasoline. In this embodiment, input data may include historical laboratory measurements of the density, distillation cuts, and flash points and/or the compositional analysis (concentration of methane, ethane, propane, butanes, pentanes, inert gases, average molecular weight) of the gasoline stream, compositional analysis of the butane stream (isobutane:n-butane ratio, residual propane and ethane content), and flow rate and density readings from in-line analyzers measuring both streams. In some embodiments, statistical models are used to estimate the distillation cuts, average molecular masses, and flash points of the gasoline and butane streams before the blend.

In yet another exemplary embodiment of a virtual analyzer, vapor pressure may be predicted for the blending of condensate into crude oil. In this embodiment, input data may include historical laboratory measurements of the density and vapour pressure and/or compositional analysis (concentration of methane, ethane, propane, butanes, pentanes, inert gases, average molecular weight) of the crude oil and condensate streams, and flow rate and density readings from in-line analyzers measuring both streams. In some embodiments, statistical models are used to estimate the live vapor pressure of the crude oil and condensate streams before the blend.

In yet another exemplary embodiment of a virtual analyzer, distillation cuts may be predicted for the blending of a combination of substances to meet a specific oil grade (for example, West Texas Intermediate). The distillation cuts can include residuals, light ends, the 50% point, and so forth. The combination of substances can include crude oils, natural gas liquids (including butane), condensate, and/or refinery cuts such as gasoil, residuals, etc. In this embodiment, input data may include historical laboratory measurements of the density and distillation cuts (actual or simulated) and/or the compositional analysis (concentration of methane, ethane, propane, butanes, pentanes, inert gases, average molecular weight) of each of the substances, and flow rate and density readings from in-line analyzers measuring all input substance streams. In some embodiments, statistical models are used to estimate the average molecular mass and the distillation cuts of the substance streams before the blend.

In yet another exemplary embodiment of a virtual analyzer, total acid number may be predicted for the blending of a combination of substances to meet a specific oil grade. The combination of substances can include crude oils, natural gas liquids (including butane), condensate, and/or refinery cuts such as gasoil, residuals, etc. In this embodiment, input data may include historical laboratory measurements of the density, viscosity, distillation cuts, and/or the compositional analysis (concentration of methane, ethane, propane, butanes, pentanes, inert gases, average molecular weight, total acid number) of each of the substances, and flow rate and density and viscosity readings from in-line analyzers measuring all input substance streams. In some embodiments, statistical models are used to estimate the viscosity and total acid number of the substance streams before the blend.

In yet another exemplary embodiment of a virtual analyzer, nickel and/or vanadium content may be predicted for the blending of a combination of substances to meet a specific oil grade. The combination of substances can include crude oils, natural gas liquids (including butane), condensate, and/or refinery cuts such as gasoil, residuals, etc. In this embodiment, input data may include historical laboratory measurements of the density, sulphur, viscosity, and/or the compositional analysis (concentration of methane, ethane, propane, butanes, pentanes, inert gases, average molecular weight, nickel, vanadium, total acid number, asphaltene content, micro-carbon residue) of each of the substances, and flow rate and density readings from in-line analyzers measuring all input substance streams. In some embodiments, statistical models are used to estimate the total acid number and viscosity of the substance streams before the blend.

In yet another exemplary embodiment of a virtual analyzer, parameters such as compatibility, solubility blending number, and p-value may be predicted for the blending of a combination of substances to meet a specific oil grade. The combination of substances can include crude oils, natural gas liquids (including butane), condensate, and/or refinery cuts such as gasoil, residuals, etc. In this embodiment, input data may include historical laboratory measurements of the density, solubility blending number, insolubility number, p-value, distillation cuts, and/or the compositional analysis (concentration of methane, ethane, propane, butanes, pentanes, inert gases, average molecular weight, asphaltene content, micro-carbon residue, sulphur, naphthene, aromatics content, parrafins) of each of the substances, and flow rate and density readings from in-line analyzers measuring all input substance streams. In some embodiments, statistical models are used to estimate the solubility blending number, p-value, and distillation cuts of the substance streams before the blend.

In yet another exemplary embodiment of a virtual analyzer, sulphur content may be predicted for the blending of a combination of substances to meet a specific oil grade. The combination of substances can include natural gas liquids (including butane), and refinery cuts such as gasoil, residuals, etc. In this embodiment, input data may include historical laboratory measurements of the density and/or the compositional analysis (concentration of methane, ethane, propane, butanes, pentanes, inert gases, average molecular weight, sulphur content) of each of the substances, and flow rate and density readings from in-line analyzers measuring all input substance streams. In some embodiments, statistical models are used to estimate the sulphur content of the substance streams before the blend.

In yet another exemplary embodiment of a virtual analyzer, viscosity may be predicted for the blending of a combination of substances to meet a specific oil grade. The combination of substances can include natural gas liquids (including butane), and refinery cuts such as gasoil, residuals, etc. In this embodiment, input data may include historical laboratory measurements of the density, viscosity, and distillation cuts (actual or simulated) and/or the compositional analysis (concentration of methane, ethane, propane, butanes, pentanes, inert gases, average molecular weight) of each of the substances, and flow rate and density readings from in-line analyzers measuring all input substance streams. In some embodiments, statistical models are used to estimate the average molecular mass, distillation cuts, and viscosities of the substance streams before the blend.

In yet another exemplary embodiment of a virtual analyzer, flash point may be predicted for the blending of a combination of substances to meet a specific oil grade. The combination of substances can include natural gas liquids (including butane), and refinery cuts such as gasoil, residuals, etc. In this embodiment, input data may include historical laboratory measurements of the density, distillation cuts (actual or simulated), flash points, and/or the compositional analysis (concentration of methane, ethane, propane, butanes, pentanes, inert gases, average molecular weight) of each of the substances, and flow rate and density readings from in-line analyzers measuring all input substance streams. In some embodiments, statistical models are used to estimate the average molecular mass, distillation cuts, and flash points of the substance streams before the blend.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1-20. (canceled)

21. A method for predicting physical properties of a substance by indirect measurement, comprising:
receiving measurements from a plurality of inputs; and
calculating predictions of at least one physical property of the substance, based on the received measurements;
wherein the received measurements are measurements of properties of at least one petroleum product; and
wherein the predictions are calculated at a minimum frequency.

22. The method of claim 21, wherein the inputs include one or more of: in-line physical analyzers, in-line meters, laboratory analyses, spot samples, and laboratory information management systems.

23. The method of claim 21, further comprising adjusting parameters of a petroleum process based on the predictions.

24. The method of claim 21, wherein the predictions are calculated based on a combination of physical process models, physical property models, and statistical models.

25. The method of claim 21, wherein the received measurements include one or more of: density, viscosity, flow rate, temperature, pressure, vapour pressure, vapour/liquid ratio, compositional analysis, flash point, distillation cuts, solubility blending number, insolubility number, and p-value.

26. The method of claim 21, wherein the minimum frequency of the predictions is such that a parameter of a petroleum process is modifiable, based on the predictions, during execution of the process.

27. The method of claim 26, wherein the petroleum process is a blending process, a separation process, or a chemical modification process.

28. The method of claim 27, wherein the blending process is one of:
blending of butane into crude oil; blending of butane into condensate; blending of butane into gasoline; blending of condensate into crude oil; and blending of a combination of substances to meet a specific oil grade.

29. The method of claim 21, wherein the minimum frequency of the predictions is correlated with a timescale of a petroleum process.

30. The method of claim 21, wherein the predictions include one or more of: vapour pressure, flash point, pour point, distillation cuts, total acid number, nickel content, vanadium content, sulphur content, asphaltene content, hydrogen sulphide content, compatibility, solubility blending number, viscosity, heat of combustion, asphaltene solubility, octane rating, and p-value.

31. A system for predicting physical properties of a substance by indirect measurement, comprising:
a virtual analyzer;
a plurality of measurement inputs; and
a database;
wherein the virtual analyzer receives measurements from the measurement inputs and calculates predictions of at least one physical property of the substance, based on the received measurements;
wherein the received measurements are measurements of properties of at least one petroleum product; and
wherein the virtual analyzer calculates the predictions at a minimum frequency.

32. The system of claim 31, wherein the inputs include one or more of: in-line physical analyzers, in-line meters, laboratory analyses, spot samples, and laboratory information management systems.

33. The system of claim 31, wherein parameters of a petroleum process are adjusted based on the predictions.

34. The system of claim 31, wherein the predictions are calculated based on a combination of physical process models, physical property models, and statistical models.

35. The system of claim 31, wherein the received measurements include one or more of: density, viscosity, flow rate, temperature, pressure, vapour pressure, vapour/liquid ratio, compositional analysis, flash point, distillation cuts, solubility blending number, insolubility number, and p-value.

36. The system of claim 31, wherein the minimum frequency of the predictions is such that a parameter of a petroleum process is modifiable, based on the predictions, during execution of the process.

37. The system of claim 36, wherein the petroleum process is a blending process, a separation process, or a chemical modification process.

38. The system of claim 37, wherein the blending process is one of: blending of butane into crude oil; blending of butane into condensate; blending of butane into gasoline; blending of condensate into crude oil; and blending of a combination of substances to meet a specific oil grade.

39. The system of claim 31, wherein the minimum frequency of the predictions is correlated with a timescale of a petroleum process.

40. The system of claim 31, wherein the predictions include one or more of: vapour pressure, flash point, pour point, distillation cuts, total acid number, nickel content, vanadium content, sulphur content, asphaltene content, hydrogen sulphide content, compatibility, solubility blending number, viscosity, heat of combustion, asphaltene solubility, octane rating, and p-value.
